(19) United States
(12) Patent Application Publication
(10) Pub. No.: US 2025/0228462 A1
(43) Pub. Date: Jul. 17, 2025

(54) SYSTEMS AND METHODS FOR RESPONSIVE DAMPING OF PRESSURE

(71) Applicant: Becton, Dickinson and Company, Franklin Lakes, NJ (US)

(72) Inventors: Huy Thanh Vu, Westminster, CA (US); Greg Joseph Dugan, Corona, CA (US); William Allen Rodemeyer, Corona, CA (US)

(21) Appl. No.: 19/169,663

(22) Filed: Apr. 3, 2025

Related U.S. Application Data
(63) Continuation of application No. PCT/US2023/076265, filed on Oct. 6, 2023.
(60) Provisional application No. 63/378,757, filed on Oct. 7, 2022.

(51) Int. Cl. A61B 5/0235

(52) U.S. Cl. CPC A61B 5/0235

(57) ABSTRACT

Systems and methods for responsively damping pressure pulsations are provided. Generally, a responsive damping system can comprise one or more responsive dampeners. Responsive dampeners can damp pressure pulsations via a pressure resistor.

### CROSS-REFERENCE TO RELATED APPLICATIONS

The current application is a continuation of International Application No. PCT/US2023/076265, filed Oct. 6, 2023, entitled “Systems and Methods for Responsive Damping of Pressure,” which claims priority under 35 U.S.C. 119 (e) to U.S. Provisional Application No. 63/378,757, entitled “Systems and Methods for Active Damping of Pressure” to Vu et al., filed Oct. 7, 2022, the disclosures of which are herein incorporated by reference in their entireties.

### TECHNOLOGICAL FIELD

The disclosure is generally directed to systems and methods for responsive damping of pressure, and more specifically for responsive damping pressure for use in blood pressure monitoring systems.

### BACKGROUND

Continuous noninvasive blood pressure monitors enable real-time measurement of blood pressure waves and derived hemodynamic parameters. Multiple techniques can be utilized including the volume clamp method.

Volume clamp method measures arterial blood pressure at an extremity (e.g., finger) utilizing an inflatable cuff, a light source (e.g., light emitting diode (LED)), and light sensor. The pressure in the cuff is adjusted to keep the diameter of the artery constant (the unloaded state), in which the diameter is determined via the light source and light sensor. The pressure within the inflatable cuff represents the arterial pressure of the finger artery. A pressure pump supplies the pressure to the inflatable cuff.

### SUMMARY

Systems and methods for responsive damping pressure pulsations utilize a responsive damping system that comprises a set of one or more responsive dampeners. Each responsive dampener can comprise a mechanism for providing a resistant force that is responsive to the undulations of the pressure supplied. The dampened pressure can be utilized in various medical devices, such as a blood pressure monitoring system.

In some implementations, a responsive damping system for damping pressure pulsations from a pressure supply source. The responsive damping system comprises an inlet, an outlet, and a set of one or more responsive dampeners that are in fluidic connection.

In some implementations, each responsive dampener comprises a poppet in connection with a resistor.

In some implementations, the poppet comprises a head that is in contact a valve seat.

In some implementations, the poppet can move bidirectionally between a front end and a back end of each responsive dampener.

In some implementations, the bidirectional movement of the poppet is based on an amount of pressure flow present and the amount of resistance force provided by the resistor.

In some implementations, each responsive dampener comprises a bellows in connection with a resistor.

In some implementations, the resistor is in contact with the disc.

In some implementations, the bellows, the disc, and resistor are configured such that when flow pressure is increased and amount of resistant force provided by the bellows and resistor is increased.

In some implementations, each responsive dampener comprises a bellows in connection with an electromagnet configured to provide a resistant force.

In some implementations, the electromagnet is in contact with the disc.

In some implementations, end opposite of the inlet, wherein the electromagnet is in contact with the disc, wherein the bellows, the disc, and electromagnet are configured such that when flow pressure is increased the resistant force provided by the bellows and electromagnet is increased.

In some implementations, the electromagnet is in communication with the pressure pump and configured such that the resistant force provided by the electromagnet adjusts in accordance with the pressure generated by the pressure pump.

In some implementations, at points of higher generated pressure relative to an average pressure to be generated, increased current is provided to the electromagnet to increase resistant force provided by the electromagnet.

In some implementations, at points of lower generated pressure relative to an average pressure to be generated, decreased current is provided to the electromagnet to decrease resistant force provided by the electromagnet.

In some implementations, the bellows is configured to receive pressured air from an inlet.

In some implementations, the bellows comprises a disc at an end opposite of the inlet.

In some implementations, the responsive damping system comprises a channel connecting the bellows with an outlet.

In some implementations, the channel has a cross-sectional area that is at least 2× less than a cross-sectional area of the bellows.

In some implementations, each dampener is kept in a housing that is airtight.

In some implementations, the set of one or more responsive dampeners comprises a responsive dampener that utilizes a spring as the resistor.

In some implementations, the set of one or more responsive dampeners comprises a responsive dampener that utilizes a polymer gel as the resistor.

In some implementations, the set of one or more responsive dampeners comprises a responsive dampener that utilizes a set of magnets as the resistor.

In some implementations, the set of one or more responsive dampeners comprises at least two responsive dampeners, wherein the at least two responsive dampeners utilize a spring as the resistor.

In some implementations, the set of one or more responsive dampeners comprises at least two responsive dampeners, wherein the at least two responsive dampeners utilize a polymer gel as the resistor.

In some implementations, the set of one or more responsive dampeners comprises at least two responsive dampeners, wherein the at least two responsive dampeners utilize a set of magnets as the resistor.

In some implementations, the set of one or more responsive dampeners comprises at least two responsive dampeners, wherein a first responsive dampener of the at least two responsive dampeners utilizes a spring as the resistor and a second responsive dampener of the at least two responsive dampeners utilizes a polymer gel as the resistor.

In some implementations, the set of one or more responsive dampeners comprises at least two responsive dampeners, wherein a first responsive dampener of the at least two responsive dampeners utilizes a spring as the resistor and a second responsive dampener of the at least two responsive dampeners utilizes a set of magnets as the resistor.

In some implementations, the set of one or more responsive dampeners comprises at least two responsive dampeners, wherein a first responsive dampener of the at least two responsive dampeners utilizes a polymer gel as the resistor and a second responsive dampener of the at least two responsive dampeners utilizes a set of magnets as the resistor.

In some implementations, the set of one or more responsive dampeners comprises at least three responsive dampeners, wherein a first responsive dampener of the at least three responsive dampeners utilizes a spring as the resistor, wherein a second responsive dampener of the at least three responsive dampeners utilizes a polymer gel as the resistor, and a third responsive dampener of the at least three responsive dampeners utilizes a set of magnets as the resistor.

In some implementations, the responsive damping system further comprises a pressure pump in fluidic connection with the set of one or more responsive dampeners, wherein the pressure pump is the pressure supply source.

In some implementations, the pressure pump is a positive-displacement pump, a centrifugal pump, or an axial-flow pump.

In some implementations, the pressure pump is a rotary-type pump, a reciprocating-type pump, a linear-type pump, or a pneumatic pump.

In some implementations, the responsive damping system is utilized within a pressure system utilized in conjunction with a medical device.

In some implementations, the responsive damping system further comprises a blood pressure monitoring system. The blood pressure monitoring system comprises a blood pressure cuff in fluidic connection with the set of one or more responsive dampeners.

In some implementations, the blood pressure monitoring system further comprises a pressure control system that senses the pressure amount and can adjust the supplied pressure.

In some implementations, the blood pressure cuff is configured to surround an arm or a digit of a patient.

In some implementations, a method is for damping pressure pulsations from a pressure supply source via a responsive damping system for use with a blood pressure monitoring system. The method provides pressure from the pressure supply source. The method passes the provided pressure through the responsive dampening system. The responsive damping system comprises an inlet, an outlet, and a set of one or more responsive dampeners that are in fluidic connection. Each responsive dampener comprises a resistor. The method passes the dampened pressure to a blood pressure cuff.

In some implementations, the method further passes the dampened pressure through a pressure sensor for measuring a pressure level the dampened pressure.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1 provides a mock example of data graph of pressure showing the pressure undulations as generated by a pressure source.

FIG. 2 provides an exemplary blood pressure monitoring system incorporating a responsive damping system.

FIGS. 3A and 3B provide an example of a responsive dampener that utilizes a poppet and a spring. FIG. 3A provides a perspective view and FIG. 3B provides a cross-sectional view.

FIGS. 3C and 3D provide an example of a responsive dampener that utilizes a poppet and a polymer gel. FIG. 3C provides a perspective view and FIG. 3D provides a cross-sectional view.

FIGS. 3E and 3F provide an example of a responsive dampener that utilizes a poppet and a set of magnets. FIG. 3E provides a perspective view and FIG. 3F provides a cross-sectional view.

FIGS. 4A and 4B provide an example of a responsive dampener that utilizes a bellows and a spring. FIG. 4A provides an exploded view and FIG. 4B provides a cross-sectional view.

FIGS. 5A and 5B provide an example of a responsive dampener that utilizes a bellows and a electromagnet. FIG. 5A provides an exploded view and FIG. 5B provides a cross-sectional view.

FIGS. 6A to 6D provide an example of a responsive damping system. FIG. 6A provides a perspective view, FIG. 6B provides a frontal view, FIG. 6C provides an exploded view, and FIG. 6D provides a cross-sectional view.

### DETAILED DESCRIPTION

The current disclosure details systems and methods for responsively damping pressure in a blood pressure monitoring system or other medical devices that utilize a pressure source (also referred to as actively damping). Pressure pumps and other pressure sources typically provide undulating pressure, and although the undulation is minor, it can cause inaccuracies in sensitive measurements and treatments. FIG. 1 provides a simulated example of a data graph of a pressure measurement that is typical of a pressure source providing a constant pressure. While the average pressure 101 provided is constant, the actual provided pressure 103 undulates up and down, yielding a pulsation effect of the average provided pressure. The pulsation effect is due to the functioning the pump as it draws in air. For instance, in a pneumatic pump, the amount of pressure provided undulates in accordance with the piston/plunger moving back and forth.

It is a goal of the current application to reduce the pulsation effect that is provided by a pressure source via a responsive damping system. A responsive damping system can comprise a means for responsively damping the pressure undulation into a more smoothened provided pressure, with less pulsation. The smoothened pressure can yield better accuracy when used in a blood pressure monitoring system or other medical device that benefits from less pressure pulsation.

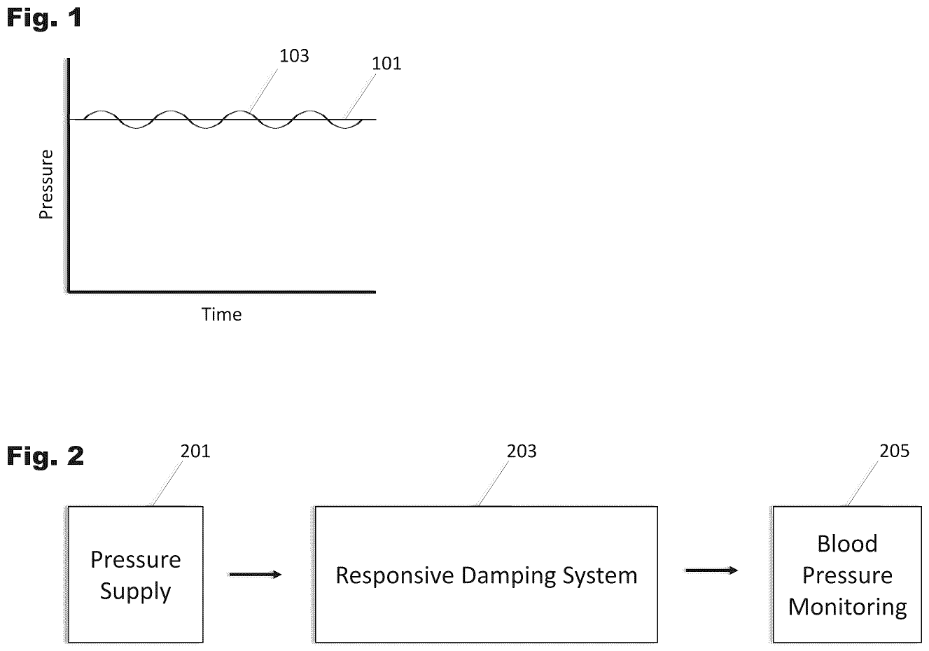

Provided in FIG. 2 is an overview system diagram of a pressure system utilized within a blood pressure monitoring system. The pressure system comprises a pressure supply 201 and a responsive damping system 203. Pressure supply 201 can be any source that can provide pressure, such a pressure pump. The pressure pump can be a positive-displacement pump, a centrifugal pump, an axial-flow pump or any other pump capable of generating pressure. In some instances, the pressure pump is a positive-displacement pump. Types of positive-displacement pumps that can be utilized include (but are not limited to) rotary-type pumps, reciprocating-type pumps, linear-type pumps, and pneumatic pumps. The various pumps each provide a relative amount of pressure undulation as the pump mechanics facilitate the movement of fluid (liquid or gas).

The pressure provided by the pressure supply 201 is transferred through a responsive damping system 203 to reduce the amount of pulsation of the pressure as provided by the supply. Responsive damping system 203 can comprise a set of one or more responsive dampeners that provide a means for damping pressure undulation. Generally, each responsive dampener provides a resistance to the pressure that is adjustable based on the immediate pressure level traveling through the dampener.

In some instances, a responsive dampener comprises a poppet valve with a resistor that allows the poppet valve to provide more resistance as the pressure and/or flow rate is increased. Resistors can be any structure or material that can increase resistance as a poppet valve experiences greater pressure and/or flow rate. Examples of resistors include (but are not limited to) a spring, a set of two or magnets, a polymer gel, and an electromagnet.

In some instances, a responsive dampener comprises an electromagnet in connection with a resistor that can provide a resistance level based upon an amount of electrical current. The electromagnet can be in communication with the pump source such that the electromagnet can respond as the pump works to provide pressure. In moments when greater pressure is expected, greater electrical current is generated to provide greater magnetic resistance.

The responsive damping system can be combined with one or more passive damping systems, which can be provided before, after, or integrated within the responsive damping system. Typically, passive damping systems utilize expansion chambers and/or alter direction of flow to damp pressure undulation. In some instances, a responsive damper is provided within an expansion chamber. In some instances, a responsive damper is provided within a bellows chamber, which can expand and contract as pressurized air pass through. A filter can also be provided before, after, or integrated within the responsive damping system.

When responsive damping system 203 is utilized within a blood pressure monitoring system, the pressure outflow out of the responsive damping system is utilized for blood pressure monitoring 205. Generally, the dampened pressure outflow travels through a pressure control system that comprises a pressure sensor to sense the amount of pressure and can adjust the supplied pressure as necessary. The pressure is then transferred into a blood pressure cuff, which can be a cuff surrounding any extremity of a patient, such as (for example) an arm or a digit. In some instances, the blood pressure monitoring system utilizes a volume clamp method for continuous blood pressure monitoring and thus the pressure is adjusted based on the amount of pressure to keep the diameter of a patient's artery constant via the pressure cuff. When a volume clamp method is utilized, the supplied pressure to the cuff that keeps the artery diameter constant is the blood pressure within that artery.

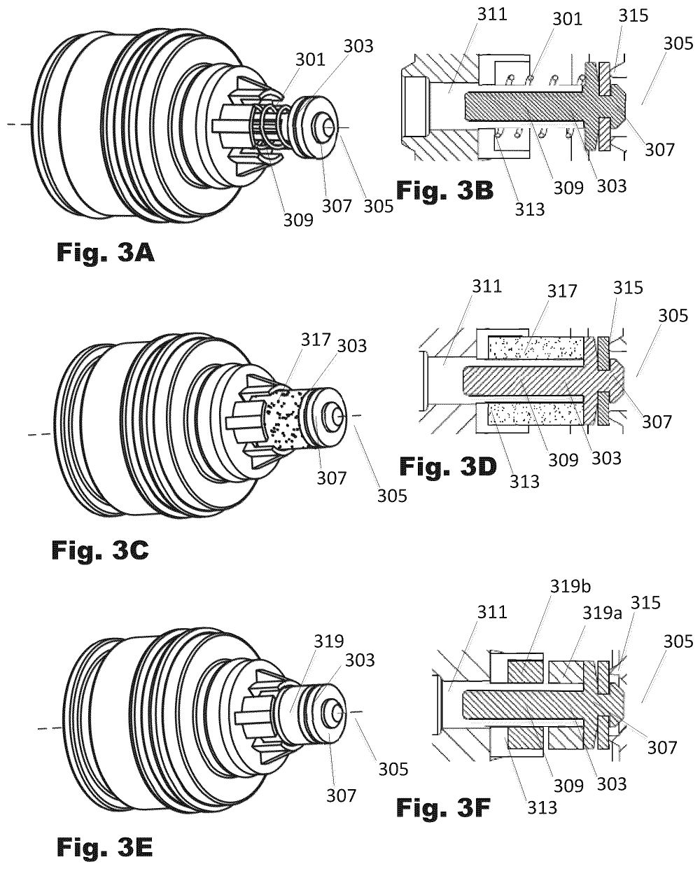

Responsive dampeners comprise a means to resist pressure pulsations. In many instances, pressure pulsations are responsively dampened via a poppet in combination with a spring, a set of two or magnets, and a polymer gel. Provided in FIGS. 3A to 3F are examples of responsive dampeners that can be utilized within a responsive damping system.

FIGS. 3A and 3B provide an example of a responsive dampener using a spring 301 to provide a resistant force to smoothen pressurized air. A poppet 303 is provided at a front end 305 and comprises a head 307 and a rod 309 that allows the poppet move bidirectionally between front end 305 and a back end 311. Spring 301 can encircle rod 309 and is in contact with head 307 and base wall 313. When no pressure flow is present, poppet head 307 is in contact with valve seat 315 resulting in the poppet valve being closed. As pressure flows through front end 305, the pressure flow pushes poppet head 307 towards back end 311 and spring 301 provides resistance to poppet head 307 and the pressure flow such that as flow pressure is increased the resistant force provided by the spring is increased. The resistance provided by spring 301 responsively dampens pressure pulsation that is provided by a pressure source. Smoothened pressurized air can travel past the poppet and onward towards back end 311. The responsive dampener can be kept in a housing that is airtight such that it can transfer the pressurized air therethrough.

FIGS. 3C and 3D provide an example of a responsive dampener using a polymer gel 317 to provide a resistant force to smoothen pressurized air. The responsive dampener using polymer gel 317 can have essentially the same poppet 303 as described in FIGS. 3A and 3B except the polymer gel 317 replaces spring 301. Polymer gel 317 can partially or fully surround rod 309 and is in contact with head 307 and base wall 313. Polymer gel 317 has a compressive strength to provide the resistant force as pressure flows through front end 305 and pushes poppet head 307 towards back end 311 such that as flow pressure is increased the resistant force provided by the polymer gel is increased. The resistance provided by polymer gel 317 responsively dampens pressure undulation that is provided by a pressure source. Smoothened pressurized air can travel past the poppet and onward towards back end 311. The responsive dampener can be kept in a housing that is airtight such that it can transfer the pressurized air therethrough.

FIGS. 3E and 3F provide an example of a responsive dampener using a set of magnets 319 to provide a resistant force to smoothen pressurized air. The responsive dampener using set of magnets 319 can have essentially the same poppet 303 as described in FIGS. 3A and 3B except the set of magnets 319 replaces spring 301. Set of magnets 319 can partially or fully surround rod 309 and a first magnet 319a is in contact with head 307 and a second magnet 319b is contact base wall 313 such that the adjacent faces of magnet 319a and magnet 319b have opposite poles. The force of the opposite poles of adjacent faces of magnet 319a and magnet 319b provide the resistant force as pressure flows through front end 305 and pushes poppet head 307 towards back end 311 such that as flow pressure is increased the resistant force provided by the magnets is increased. The resistance provided by set of magnets 319 responsively dampens pressure pulsation that is provided by a pressure source. Smoothened pressurized air can travel past the poppet and onward towards back end 311. The responsive dampener can be kept in a housing that is airtight such that it can transfer the pressurized air therethrough.

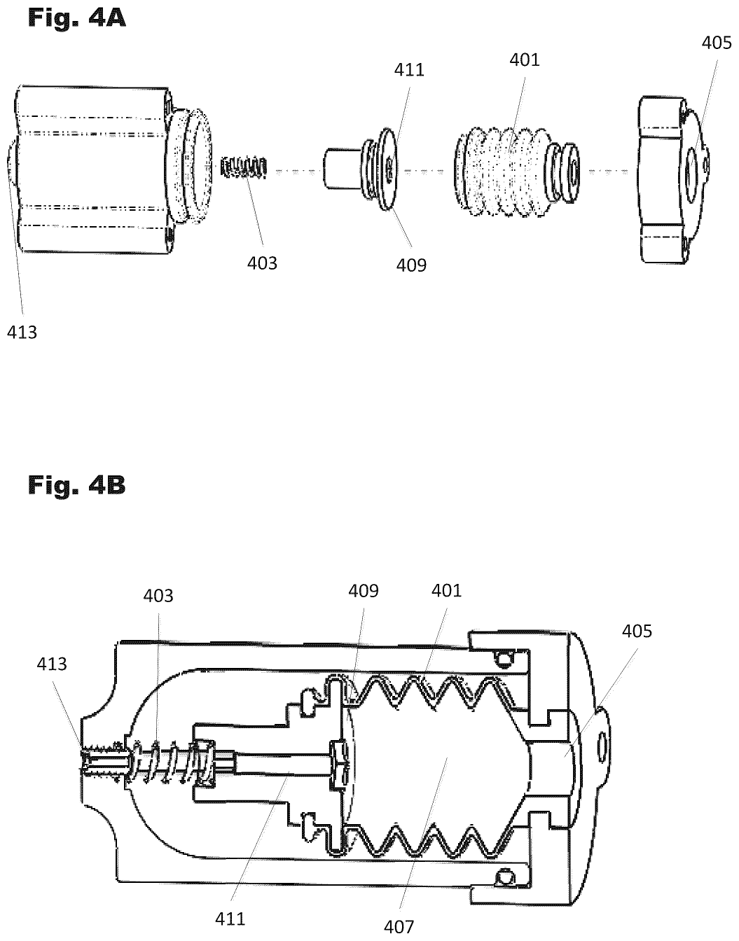

FIGS. 4A and 4B provide an example of a responsive dampener using a bellows and spring, which can respond to pressure as it passes through. A bellows 401 can work with a spring 403 to provide a resistant force to smoothen pressurized air. The responsive dampener includes an inlet 405 to allow pressurized air to enter and expand with bellows chamber 407. The pressurized air comes in contact with a disc 409 that is the backend of the bellows chamber 407 and is further in contact with spring 403. Disc 409, bellows 401 and spring 403 are configured such that when flow pressure is increased the resistant force provided by the bellows and spring is increased. The resistance provided by set of bellows 401 and spring 403 responsively dampens pressure pulsation that is provided by a pressure source. Disc 409 can include a channel 411 to allow the smoothened pressurized air transfer therethrough and out of an outlet 413. Channel 411 can have a cross-sectional area that is less than the cross-sectional area of bellows chamber 407 (e.g., at least 2× less cross-area). The responsive dampener can be kept in a housing that is airtight such that it can transfer the pressurized air therethrough. Although a spring is depicted as a means to provide a resistant force, any resistor can be utilized, including (but not limited to) a polymer gel and a set of magnets.

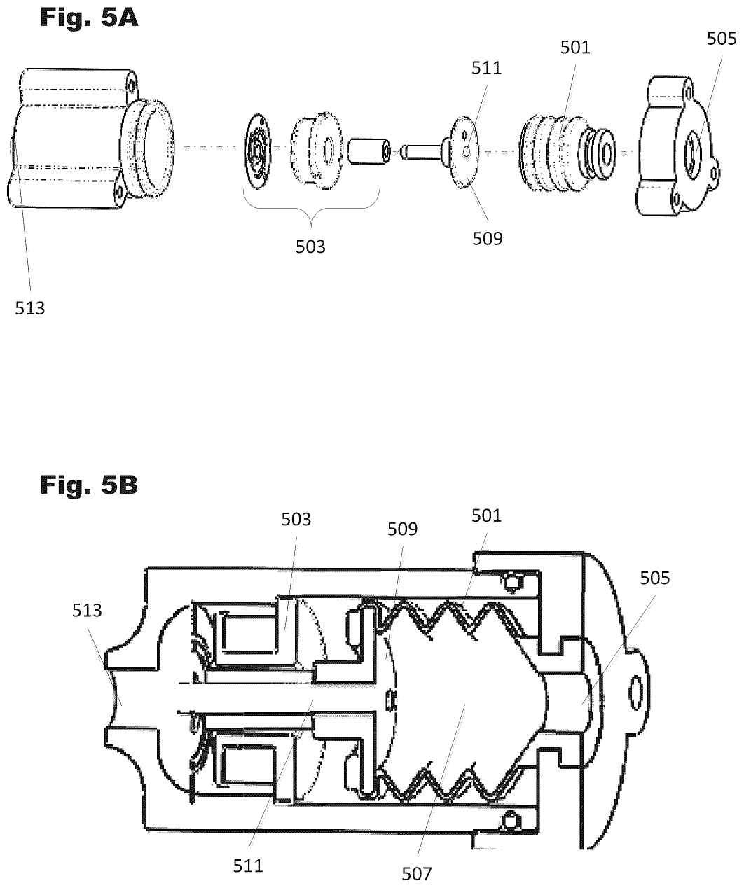

FIGS. 5A and 5B provide an example of a responsive dampener using a bellows and electromagnet, which can respond to pressure as it passes through. A bellows 501 can work with a electromagnet 503 to provide a resistant force to smoothen pressurized air. The responsive dampener includes an inlet 505 to allow pressurized air to enter and expand with bellows chamber 507. The pressurized air comes in contact with a disc 509 that is the backend of the bellows chamber 507 and is further in contact with electromagnet 503. Electromagnet 503 can increase and decrease its magnetic force with current provided. Electromagnet 503 can have an electrical power source that provides a current that is aligned with the mechanics of the pressure supply source. As discussed in reference to FIG. 2, pressure pumps generate undulating pressure relative to the average pressure generated. At points of higher generated pressure relative to the average pressure to be generated, the current provided to electromagnet 503 is increased to increase the magnetic force and thus increasing the resistant force. And at points of lower generated pressure relative to the average pressure to be supplied, the current provided to electromagnet 503 is decreased to decrease the magnetic force and thus decreasing the resistant force. Thus, when the pressurized air contacts disc 509, the resistant force provided by electromagnet 503 has been adjusted accordingly to smoothen the pressure pulsations. Disc 509 can include a channel 511 to allow the smoothened pressurized transfer therethrough and out of an outlet 513. Channel 511 can have a cross-sectional area that is less than the cross-sectional area of bellows chamber 507 (e.g., at least 2× less cross-area). The responsive dampener can be kept in a housing that is airtight such that it can transfer the pressurized air therethrough.

A responsive damping system comprises a set of one or more responsive dampeners. The responsive damping system includes an inlet and outlet and the set of one or more responsive dampeners is provided therebetween such that a pressure flow can transfer through the set of responsive dampeners. In some instances, the responsive dampeners are provided in a series such that the pressure flow sequentially passes through each of the responsive dampeners. The responsive damping system can comprise one or more types of responsive dampeners, where the type of responsive dampener is determined by the structure or material providing the resistant force (e.g., spring, polymer gel, set of magnets, electromagnet, etc.). In some instances, a set of two or more responsive dampeners utilizes two or more types of responsive dampeners. In some instances, a set of two or more responsive dampeners utilizes the same type of responsive dampener sequentially repeated.

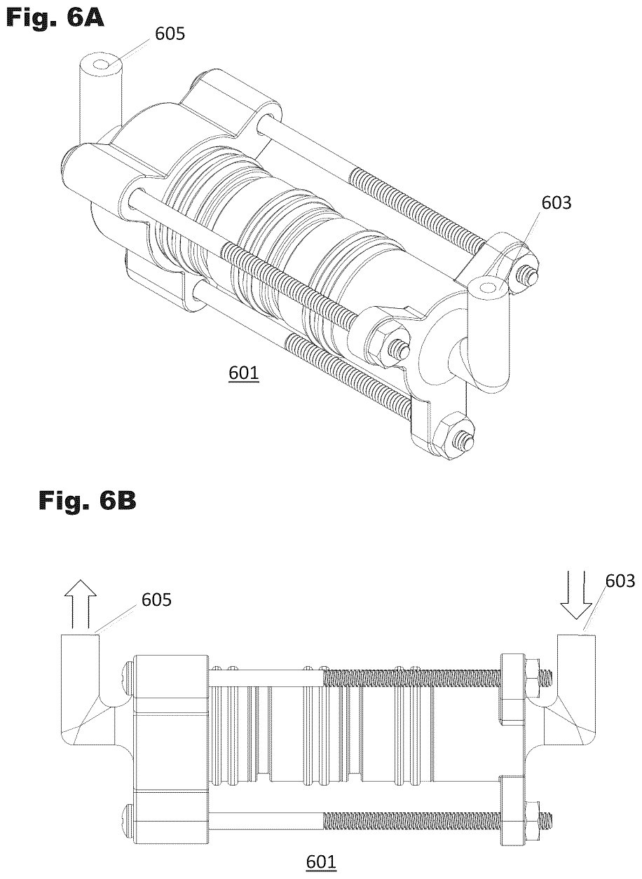

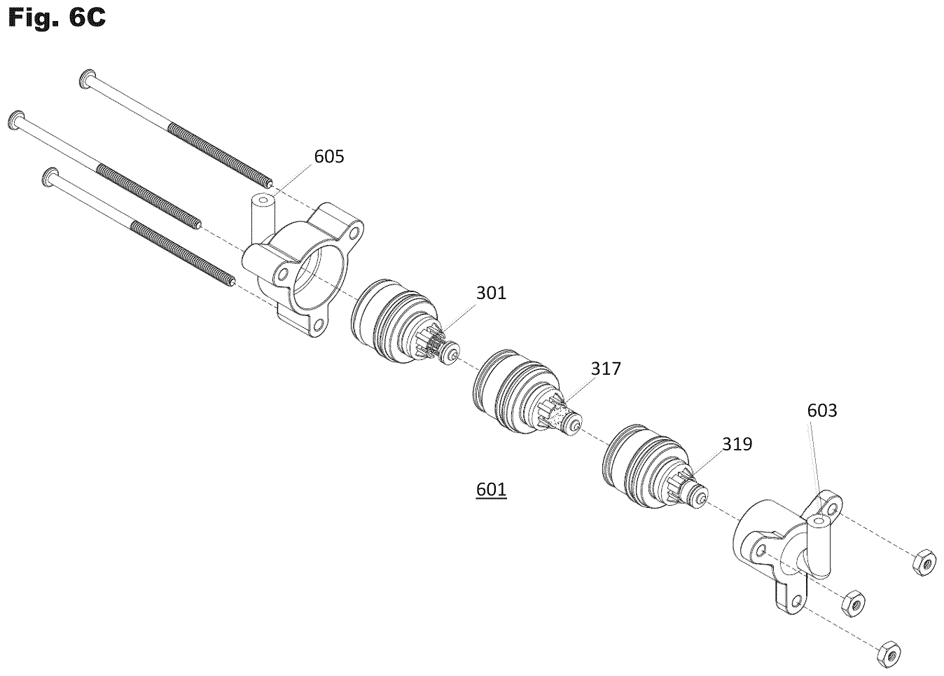

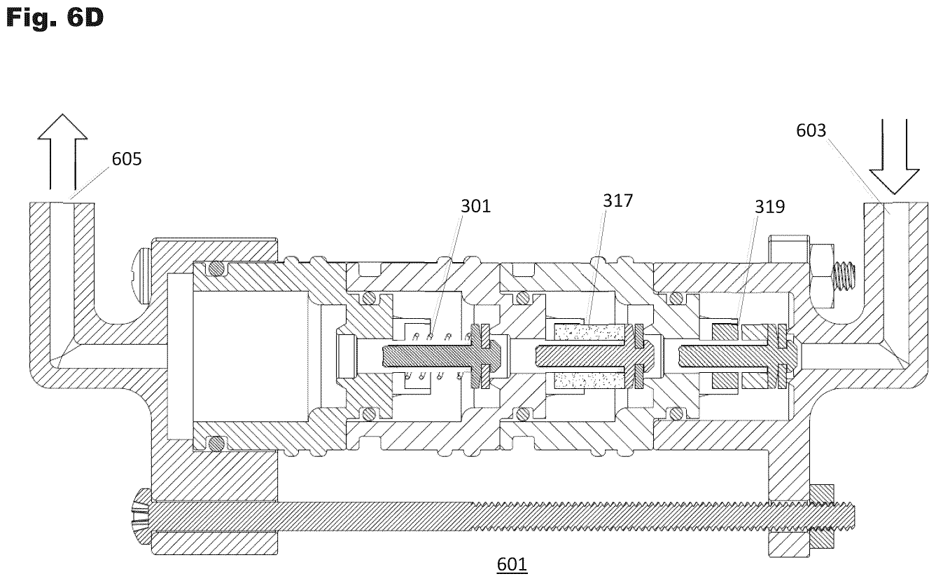

FIGS. 6A to 6D provide an example of a responsive damping system 601 for damping pressure undulations from a pressure source. responsive damping system 601 comprises a set of three responsive dampeners provided in a series that are in fluidic connection. The system comprises a responsive dampener that utilizes spring 301 for providing the resistant force, a responsive dampener that utilizes polymer gel 317 for providing the resistant force, and a responsive dampener that utilizes set of magnets 319 for providing the resistant force. Although responsive damping system 601 utilizes these types of responsive dampeners in a series with a particular order, it is to be understood that the depicted responsive damping system is an example and that a responsive damping system can utilize any combination of responsive dampeners, which can be provided in any sequential order. Further, it should be understood that the dampeners described in FIGS. 4A to 5B could be utilized in addition to or in lieu of the dampeners portrayed.

In some implementations, a responsive damping system comprises a set of one or more dampeners, wherein the set of one or more dampeners comprises a dampener that utilizes a spring for providing a resistant force. In some implementations, a responsive damping system comprises a set of one or more dampeners, wherein the set of one or more dampeners comprises a dampener that utilizes a polymer gel for providing a resistant force. In some implementations, a responsive damping system comprises a set of one or more dampeners, wherein the set of one or more dampeners comprises a dampener that utilizes a set of magnets for providing a resistant force. In some implementations, a responsive damping system comprises a set of one or more dampeners, wherein the set of one or more dampeners comprises a dampener that utilizes an electromagnet for providing a resistant force.

Responsive damping system 601 includes an inlet 603 and an outlet 605 for receiving and releasing pressure flow, respectively. As shown, inlet 603 is in fluidic connection with the responsive dampener that utilizes set of magnets 319, which in turn is in fluidic connection with the responsive dampener that utilizes polymer gel 317, which in turn is in fluidic connection with the responsive dampener that utilizes spring 301, which in turn is in fluidic connection with outlet 605. Accordingly, a pressure flow can pass through inlet 603, then pass through the set of responsive dampeners to dampen pressure pulsations, and then pass through outlet 605. The outflow pressure is dampened and can be utilized in medical machinery or other devices that benefit from smoothened pressure.

Responsive damping system 601 can be combined with one or more passive damping systems, which can be provided before, after, or integrated within the responsive damping system. Furthermore, the outflow pressure out of responsive damping system 601 can be utilized in a blood pressure monitoring system and thus can be supplied to a pressure cuff for measuring blood pressure.

While an exemplary responsive damping system is described above with reference to FIGS. 6A to 6D, it should be readily appreciated that a responsive damping system can be implemented on any of a variety of implementations including various combinations of types of responsive dampeners and sequential orders or of responsive dampeners. For instance, responsive dampeners as shown and described within FIGS. 4A to 5B could be combined and/or replaced with any other responsive dampeners. Furthermore, the housing for responsive dampeners can take any form that allows for fluidic connection from the inlet to the set of responsive dampeners to the outlet. Accordingly, a responsive damping system should be understood as not limited to the specific responsive dampeners shown, the sequential order of responsive dampeners, the number of responsive dampeners, or the specific housing construction of responsive dampeners. Instead, a responsive damping system can be implemented in a variety of ways as long the means for providing responsive damping are included.

### EXAMPLES

Example 1. A responsive damping system for damping pressure pulsations from a pressure supply source, comprising:
an inlet, an outlet, and a set of one or more responsive dampeners that are in fluidic connection, wherein each responsive dampener comprises a poppet in connection with a resistor.

Example 2. The responsive damping system of example 1, wherein the poppet comprises a head that is in contact a valve seat; wherein the poppet can move bidirectionally between a front end and a back end of each responsive dampener; wherein the bidirectional movement of the poppet is based on an amount of pressure flow present and the amount of resistance force provided by the resistor.

Example 3. The responsive damping system of example 1 or 2, wherein the set of one or more responsive dampeners comprises a responsive dampener that utilizes a spring as the resistor.

Example 4. The responsive damping system of example 1, 2, or 3, wherein the set of one or more responsive dampeners comprises a responsive dampener that utilizes a polymer gel as the resistor.

Example 5. The responsive damping system of any one of examples 1-4, wherein the set of one or more responsive dampeners comprises a responsive dampener that utilizes a set of magnets as the resistor.

Example 6. The responsive damping system of any one of examples 1-5, wherein the set of one or more responsive dampeners comprises at least two responsive dampeners, wherein the at least two responsive dampeners utilize a spring as the resistor.

Example 7. The responsive damping system of any one of examples 1-6, wherein the set of one or more responsive dampeners comprises at least two responsive dampeners, wherein the at least two responsive dampeners utilize a polymer gel as the resistor.

Example 8. The responsive damping system of any one of examples 1-7, wherein the set of one or more responsive dampeners comprises at least two responsive dampeners, wherein the at least two responsive dampeners utilize a set of magnets as the resistor.

Example 9. The responsive damping system of any one of examples 1-8, wherein the set of one or more responsive dampeners comprises at least two responsive dampeners, wherein a first responsive dampener of the at least two responsive dampeners utilizes a spring as the resistor and a second responsive dampener of the at least two responsive dampeners utilizes a polymer gel as the resistor.

Example 10. The responsive damping system of any one of examples 1-9, wherein the set of one or more responsive dampeners comprises at least two responsive dampeners, wherein a first responsive dampener of the at least two responsive dampeners utilizes a spring as the resistor and a second responsive dampener of the at least two responsive dampeners utilizes a set of magnets as the resistor.

Example 11. The responsive damping system of any one of examples 1-10, wherein the set of one or more responsive dampeners comprises at least two responsive dampeners, wherein a first responsive dampener of the at least two responsive dampeners utilizes a polymer gel as the resistor and a second responsive dampener of the at least two responsive dampeners utilizes a set of magnets as the resistor.

Example 12. The responsive damping system of any one of examples 1-11, wherein the set of one or more responsive dampeners comprises at least three responsive dampeners, wherein a first responsive dampener of the at least three responsive dampeners utilizes a spring as the resistor, wherein a second responsive dampener of the at least three responsive dampeners utilizes a polymer gel as the resistor, and a third responsive dampener of the at least three responsive dampeners utilizes a set of magnets as the resistor.

Example 13. The responsive damping system of any one of examples 1-12 further comprising a pressure pump in fluidic connection with the set of one or more responsive dampeners, wherein the pressure pump is the pressure supply source.

Example 14. The responsive damping system of example 13, wherein the pressure pump is a positive-displacement pump, a centrifugal pump, or an axial-flow pump.

Example 15. The responsive damping system of example 13, wherein the pressure pump is a rotary-type pump, a reciprocating-type pump, a linear-type pump, or a pneumatic pump.

Example 16. The responsive damping system of any one of examples 1-15, wherein the responsive damping system is utilized within a pressure system utilized in conjunction with a medical device.

Example 17. The responsive damping system of any one of examples 1-16 further comprising a blood pressure monitoring system, wherein the blood pressure monitoring system comprises a blood pressure cuff in fluidic connection with the set of one or more responsive dampeners.

Example 18. The responsive damping system of example 17, wherein the blood pressure monitoring system further comprises a pressure control system that senses an amount of pressure amount and can adjust the supplied pressure.

Example 19. The responsive damping system of example 17, wherein the blood pressure cuff is configured to surround an arm or a digit of a patient.

Example 20. The responsive damping system of any one of examples 1-19, wherein each dampener is kept in a housing that is airtight.

Example 21. A method of damping pressure pulsations from a pressure supply source via a responsive damping system for use with a blood pressure monitoring system, the method comprising:
providing pressure from the pressure supply source;
   passing the provided pressure through the responsive damping system, wherein the responsive damping system comprises an inlet, an outlet, and a set of one or more responsive dampeners that are in fluidic connection, wherein each responsive dampener comprises a resistor; and
   passing the dampened pressure to a blood pressure cuff.

Example 22. The method of example 21 further comprising:
passing the dampened pressure through a pressure sensor for measuring a pressure level the dampened pressure.

Example 23. The method of example 21 or 22, wherein each responsive dampener comprises a poppet in connection with the resistor; wherein the poppet comprises a head that is in contact a valve seat; wherein the poppet can move bidirectionally between a front end and a back end of each responsive dampener; wherein the bidirectional movement of the poppet is based on an amount of pressure flow present and an amount of resistance force provided by the resistor.

Example 24. The method of example 21, 22, or 23, wherein each responsive dampener comprises a bellows in connection with a resistor, wherein the bellows is configured to receive pressured air from an inlet, wherein the bellows comprises a disc at an end opposite of the inlet, wherein the resistor is in contact with the disc, wherein the bellows, the disc, and resistor are configured such that when flow pressure is increased an amount of resistant force provided by the bellows and resistor is increased.

Example 25. The method of any one of examples 21-24, wherein the set of one or more responsive dampeners comprises a responsive dampener that utilizes one of: a spring as the resistor, a polymer gel as the resistor, a set of magnets as the resistor, or an electromagnet.

Example 26. A responsive damping system for damping pressure pulsations from a pressure supply source, comprising:
an inlet, an outlet, and a set of one or more responsive dampeners that are in fluidic connection, wherein each responsive dampener comprises a bellows in connection with a resistor.

Example 27. The responsive damping system of example 26, wherein the bellows is configured to receive pressured air from an inlet, wherein the bellows comprises a disc at an end opposite of the inlet, wherein the resistor is in contact with the disc, wherein the bellows, the disc, and resistor are configured such that when flow pressure is increased and amount of resistant force provided by the bellows and resistor is increased.

Example 28. The damping system of example 26 or 27, wherein the resistor is a spring.

Example 29. The damping system of example 26, 27, or 28, wherein the resistor is a polymer gel.

Example 30. The damping system of any one of examples 26-29, wherein the resistor is a set of magnets.

Example 31. The damping system of any one of examples 26-30, wherein the resistor is an electromagnet.

Example 32. The damping system of any one of examples 26-31 further comprising a channel connecting the bellows with an outlet.

Example 33. The damping system of example 32, wherein the channel has a cross-sectional area that is at least 2× less than a cross-sectional area of the bellows.

Example 34. The responsive damping system of any one of examples 26-33 further comprising a pressure pump in fluidic connection with the set of one or more responsive dampeners, wherein the pressure pump is the pressure supply source.

Example 35. The responsive damping system of example 34, wherein the pressure pump is a positive-displacement pump, a centrifugal pump, an axial-flow pump, a rotary-type pump, a reciprocating-type pump, a linear-type pump, or a pneumatic pump.

Example 36. The responsive damping system of any one of examples 26-35, wherein the responsive damping system is utilized within a pressure system utilized in conjunction with a medical device.

Example 37. The responsive damping system of any one of examples 26-36 further comprising a blood pressure monitoring system, wherein the blood pressure monitoring system comprises a blood pressure cuff in fluidic connection with the set of one or more responsive dampeners.

Example 38. The responsive damping system of example 37, wherein the blood pressure monitoring system further comprises a pressure control system that senses an amount of pressure amount and can adjust the supplied pressure.

Example 39. The responsive damping system of example 37, wherein the blood pressure cuff is configured to surround an arm or a digit of a patient.

Example 40. The responsive damping system of any one of examples 26-39, wherein each dampener is kept in a housing that airtight.

Example 41. A responsive damping system for damping pressure pulsations from a pressure supply source, comprising:

an inlet, an outlet, and a set of one or more responsive dampeners that are in fluidic connection, wherein each responsive dampener comprises a bellows in connection with an electromagnet configured to provide a resistant force.

Example 42. The responsive damping system of example 41, wherein the bellows is configured to receive pressured air from an inlet, wherein the bellows comprises a disc at an end opposite of the inlet, wherein the electromagnet is in contact with the disc, wherein the bellows, the disc, and electromagnet are configured such that when flow pressure is increased the resistant force provided by the bellows and electromagnet is increased.

Example 43. The damping system of example 41 or 42 further comprising a pressure pump in fluidic connection with the set of one or more responsive dampeners, wherein the pressure pump is the pressure supply source.

Example 44. The damping system of example 43, wherein the electromagnet is in communication with the pressure pump and configured such that the resistant force provided by the electromagnet adjusts in accordance with the pressure generated by the pressure pump.

Example 45. The damping system of example 44, wherein at points of higher generated pressure relative to an average pressure to be generated, increased current is provided to the electromagnet to increase resistant force provided by the electromagnet.

Example 46. The damping system of example 44, wherein at points of lower generated pressure relative to an average pressure to be generated, decreased current is provided to the electromagnet to decrease resistant force provided by the electromagnet.

Example 47. The responsive damping system of any one of examples 43-46, wherein the pressure pump is a positive-displacement pump, a centrifugal pump, or an axial-flow pump.

Example 48. The responsive damping system of any one of examples 43-46, wherein the pressure pump is a rotary-type pump, a reciprocating-type pump, a linear-type pump, or a pneumatic pump.

Example 49. The responsive damping system of any one of examples 41-48, wherein the responsive damping system is utilized within a pressure system utilized in conjunction with a medical device.

Example 50. The responsive damping system of any one of examples 41-49 further comprising a blood pressure monitoring system, wherein the blood pressure monitoring system comprises a blood pressure cuff in fluidic connection with the set of one or more responsive dampeners.

Example 51. The responsive damping system of example 50, wherein the blood pressure monitoring system further comprises a pressure control system that senses an amount of pressure amount and can adjust the supplied pressure.

Example 52. The responsive damping system of example 50, wherein the blood pressure cuff is configured to surround an arm or a digit of a patient.

Example 53. The responsive damping system of any one of examples 41-52, wherein each dampener is kept in a housing that airtight.

Example 54. The damping system of any one of examples 41-53 further comprising a channel connecting the bellows with an outlet.

Example 55. The damping system of example 54, wherein the channel has a cross-sectional area that is at least 2× less than a cross-sectional area of the bellows.

What is claimed is:

1. A responsive damping system for damping pressure pulsations from a pressure supply source, comprising:
an inlet, an outlet, and a set of one or more responsive dampeners that are in fluidic connection, wherein each responsive dampener comprises a bellows in connection with an electromagnet configured to provide a resistant force.

2. The responsive damping system of claim 1, wherein the bellows is configured to receive pressured air from an inlet, wherein the bellows comprises a disc at an end opposite of the inlet, wherein the electromagnet is in contact with the disc, wherein the bellows, the disc, and the electromagnet are configured such that when flow pressure is increased the resistant force provided by the bellows and the electromagnet is increased.

3. The damping system of claim 1, and further comprising a pressure pump in fluidic connection with the set of one or more responsive dampeners, wherein the pressure pump is the pressure supply source.

4. The damping system of claim 3, wherein the electromagnet is in communication with the pressure pump and configured such that the resistant force provided by the electromagnet adjusts in accordance with the pressure generated by the pressure pump.

5. The damping system of claim 4, wherein at points of higher generated pressure relative to an average pressure to be generated, the amount of current that is provided to the electromagnet increases the resistant force provided by the electromagnet.

6. The damping system of claim 4, wherein at points of lower generated pressure relative to an average pressure to be generated, the amount of current that is provided to the electromagnet decreases the resistant force provided by the electromagnet.

7. The responsive damping system of claim 3, wherein the pressure pump is a positive-displacement pump, a centrifugal pump, or an axial-flow pump.

8. The responsive damping system of claim 3, wherein the pressure pump is a rotary-type pump, a reciprocating-type pump, a linear-type pump, or a pneumatic pump.

9. The responsive damping system of claim 1, wherein the responsive damping system is utilized within a pressure system utilized in conjunction with a medical device.

10. The responsive damping system of claim 1, and further comprising a blood pressure monitoring system, wherein the blood pressure monitoring system comprises a blood pressure cuff in fluidic connection with the set of one or more responsive dampeners.

11. The responsive damping system of claim 10, wherein the blood pressure monitoring system further comprises a pressure control system that senses an amount of pressure amount and can adjust the supplied pressure.

12. The responsive damping system of claim 10, wherein the blood pressure cuff is configured to surround an arm or a digit of a patient.

13. The responsive damping system of claim 1, wherein each dampener is kept in a housing that is airtight.

14. The responsive damping system of claim 1, and further comprising a channel connecting the bellows with the outlet.

15. The responsive damping system of claim 14, wherein the channel has a cross-sectional area that is at least 2× less than a cross-sectional area of the bellows.

16. A method of damping pressure pulsations from a pressure supply source via a responsive damping system for use with a blood pressure monitoring system, the method comprising:
providing pressure from the pressure supply source;
passing the provided pressure through the responsive damping system, wherein the responsive damping system comprises an inlet, an outlet, and a set of one or more responsive dampeners that are in fluidic connection, wherein each responsive dampener comprises a resistor; and
passing the dampened pressure to a blood pressure cuff.

17. The method of claim 16 further comprising:
passing the dampened pressure through a pressure sensor for measuring a pressure level the dampened pressure.

18. The method of claim 16, wherein at least one responsive dampener comprises a bellows in connection with a resistor, wherein the bellows is configured to receive pressured air from an inlet, wherein the bellows comprises a disc at an end opposite of the inlet, wherein the resistor is in contact with the disc, wherein the bellows, the disc, and the resistor are configured such that when flow pressure is increased an amount of resistant force provided by the bellows and the resistor is increased.

19. The method of claim 18, wherein the resistor is an electromagnet and the pressure supply source is a pressure pump in fluidic connection with the set of one or more responsive dampeners; wherein the electromagnet is in communication with the pressure pump and configured such that the resistant force provided by the electromagnet adjusts in accordance with the pressure generated by the pressure pump.

20. The method of claim 19 further comprising:
determining that pressure generated is higher or is lower than an average pressure to be generated; and
providing a current to electromagnet to adjust the resistant force provided based on whether the pressure generated relative to the average pressure to be generated.
